(19) United States
(12) Patent Application Publication
(10) Pub. No.: US 2025/0361267 A1
(43) Pub. Date: Nov. 27, 2025

(54) CHIMERIC COMPOUND FOR DEGRADING CYCLOPHILIN A, PREPARATION METHOD THEREFOR, AND USE THEREOF

(71) Applicant: INSTITUTE OF MICROBIOLOGY, CHINESE ACADEMY OF SCIENCES, Beijing (CN)

(72) Inventors: Lei SUN, Beijing (CN); Wenjun LIU, Beijing (CN); Wenxian YANG, Beijing (CN); Heqiao LI, Beijing (CN); Xiaoyuan BAI, Beijing (CN)

(21) Appl. No.: 19/102,978

(22) PCT Filed: Feb. 28, 2023
(86) PCT No.: PCT/CN2023/078634

§ 371 Date: Feb. 11, 2025

(30) Foreign Application Priority Data
Aug. 11, 2022 (CN) 202210958956.7

(51) Int. Cl. C07K 5/078; A61K 38/00; A61P 29/00

(52) U.S. Cl. CPC C07K 5/06139; A61P 29/00; A61K 38/00

(57) ABSTRACT

Disclosed are a chimeric compound for degrading cyclophilin A, a preparation method therefor, and use thereof. The structural Formula of the chimeric compound is represented by Formula I. The compound represented by Formula I provided by the present invention can be used for preventing and/or treating CypA-mediated diseases, such as CypA-mediated inflammation, autoimmune diseases and/or tumors. The present invention further provides a pharmaceutical composition comprising the compound represented by Formula I as an active ingredient and at least one pharmaceutically acceptable carrier, excipient and/or diluent. The compound represented by Formula I provided by the present invention can target and degrade the CypA protein, and thus can be used for preparing a drug for treating inflammation, autoimmune diseases, tumors and other related diseases. The compound represented by Formula I of the present invention has the function of significantly inhibiting virus-induced pneumonia, rheumatoid arthritis and lung cancer cell migration and infiltration.

### RELATED APPLICATIONS

The present application is a U.S. National Phase of International Application Number PCT/CN2023/078634 filed on Feb. 28, 2023, which claims priority to Chinese Application Number 202210958956.7 filed on Aug. 11, 2022.

### TECHNICAL FIELD

The present invention relates to a chimeric compound for degrading cyclophilin A, a preparation method therefor, and use thereof, which belongs to the field of biomedicine.

### BACKGROUND ART

Cyclophilin A (CypA) is a multifunctional immunomodulatory protein widely expressed in eukaryotic cells. During the inflammatory process caused by viral infection, CypA can promote the production of inflammatory cytokines by upregulating the NF-κB signaling pathway. During the development of autoimmune diseases such as rheumatoid arthritis, systemic lupus erythematosus and psoriasis, CypA can promote leukocyte migration and induce the expression of chemokines and cytokines. The expression of CypA is significantly increased in various tumor-related diseases, which is closely related to the occurrence, metastasis and prognosis of tumors. CypA is therefore an important target protein for treating inflammation, autoimmune diseases and tumors.

PROTAC (proteolysis-targeting chimera) consists of three moieties: a small-molecule ligand that recognizes the target protein, a linker and an E3 ubiquitin ligase ligand, and can directly degrade the target protein, so as to achieve the effect of treating related diseases. At present, there are no reports on CypA-targeted PROTAC drugs.

### SUMMARY OF THE INVENTION

It is an object of the present invention to provide a chimeric compound for degrading cyclophilin A (CypA), which can target and degrade CypA and can be used for treating CypA-mediated inflammation, autoimmune diseases, tumors and other related diseases.

The structural formula of the chimeric compound provided by the present invention is represented by Formula I:

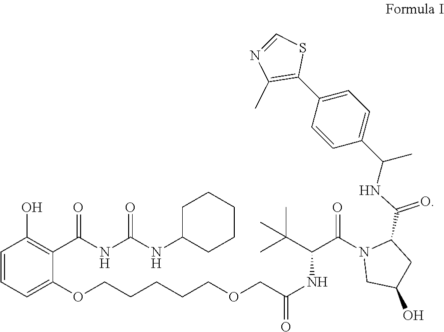 [SMILES: Cc1ncsc1-c1ccc(C(C)NC(=O)[C@@H]2C[C@@H](O)CN2C(=O)[C@H](NC(=O)COCCCCCOc2cccc(O)c2C(=O)NC(=O)NC2CCCCC2)C(C)(C)C)cc1]

The present invention further provides a preparation method for the compound represented by Formula I, comprising the steps of:
S1, subjecting a compound represented by Formula 1 to a hydrolysis reaction in the presence of a basic compound A to obtain a compound represented by Formula 2;

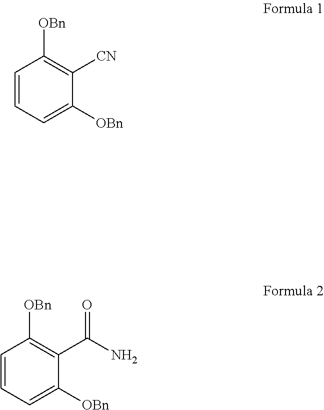

wherein Bn represents benzyl;
S2, subjecting the compound represented by Formula 2 to a nucleophilic addition reaction with a compound represented by Formula 3 to obtain a compound represented by Formula 4;

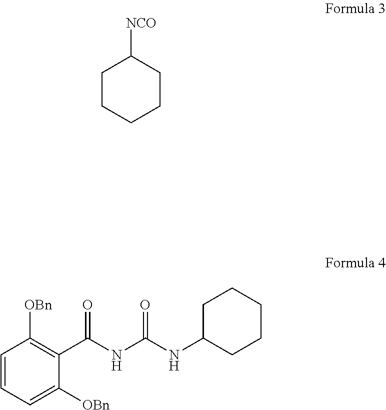

S3, reducing the compound represented by Formula 4 with palladium on carbon to obtain a compound represented by Formula 5;

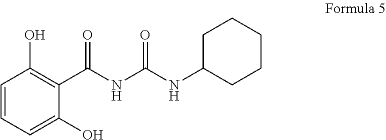 [SMILES: O=C(NC(=O)c1c(O)cccc1O)NC1CCCCC1]

S4, subjecting a compound represented by Formula 6 to a nucleophilic substitution reaction with a compound represented by Formula 7 in the presence of triethylamine and DMAP to obtain a compound represented by Formula 8;

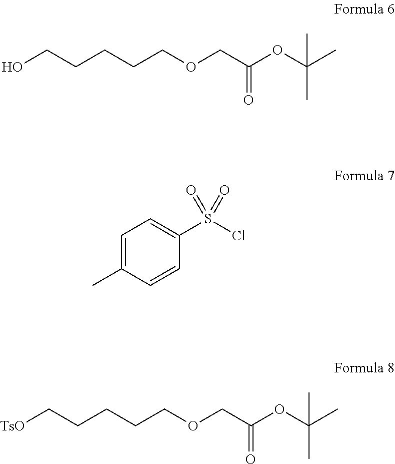

S5, subjecting the compound represented by Formula 5 to a nucleophilic substitution reaction with the compound represented by Formula 8 in the presence of a basic compound B to obtain a compound represented by Formula 9;

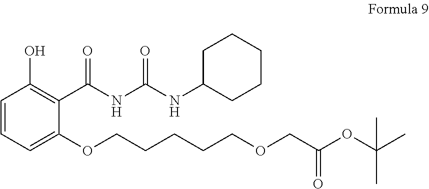 [SMILES: CC(C)(C)OC(=O)COCCCCCOc1cccc(O)c1C(=O)NC(=O)NC1CCCCC1]

S6, subjecting the compound represented by Formula 9 to a deprotection reaction in the presence of trifluoroacetic acid to obtain a compound represented by Formula 10; and

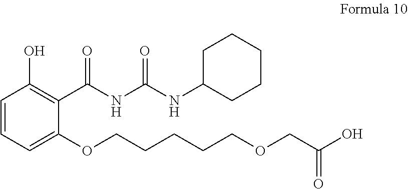 [SMILES: O=C(O)COCCCCCOc1cccc(O)c1C(=O)NC(=O)NC1CCCCC1]

S7, subjecting the compound represented by Formula 10 to an amidation reaction with a compound represented by Formula 11 in the presence of HOBT, EDCI and DIEA to obtain the compound represented by Formula I;

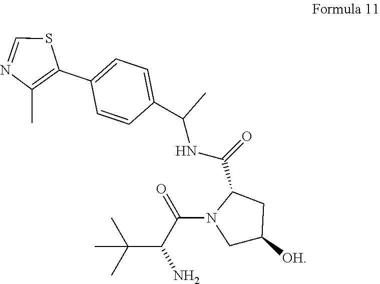 [SMILES: Cc1ncsc1-c1ccc(C(C)NC(=O)[C@@H]2C[C@@H](O)CN2C(=O)[C@H](N)C(C)(C)C)cc1]

In the above method, in step S1, the basic compound A is KOH;
a solvent for the hydrolysis reaction is benzyl alcohol, which can consume excess base in the system to inhibit further hydrolysis of amide to carboxylic acid; BnOH has poor solubility for the product, and the product precipitates directly, making it convenient for post-treatment and purification;
the molar ratio of the compound represented by Formula 1 to the benzyl alcohol is 1:5 to 10, preferably 1:6 to 7 or 1:6.87;
the temperature for the hydrolysis reaction is 100 to 150° C., preferably 130° C., and the time is 12 to 24 h, preferably 17 h.

In the above method, in step S2, the temperature for the nucleophilic addition reaction is 100 to 150° C., preferably 110° C., and the time is 12 to 24 h, preferably 17 h;
in step S3, the mass percentage content of palladium in the palladium on carbon is 10%;
the reduction reaction is carried out in methanol.

In the above method, in step S4, the molar ratio of the compound represented by Formula 6 to the compound represented by Formula 7 is 1:1 to 1.5, preferably 1:1.5; the molar ratio of the triethylamine to the compound represented by Formula 6 is 1:1 to 1.5, preferably 1:1.25;
the molar ratio of the DMAP to the compound represented by Formula 6 is 1:10 to 15, preferably 1:10;
the temperature for the nucleophilic substitution reaction is 10 to 40° C., and the time is 12 to 24 h.

In the above method, in step S5, the basic compound B is K₂CO₃;
the temperature for the nucleophilic substitution reaction is 10 to 40° C., and the time is 12 to 24 h.

In the above method, in step S6, the temperature for the deprotection reaction is 10 to 40° C., and the time is 1 to 2 h.

In the above method, in step S7, the HOBT, the EDCI and the DIEA are added to the compound represented by Formula 10 and stirred at 10 to 40° C. for 1 to 2 h, and then the compound represented by Formula 11 is added at 0° C. and reacted at 10 to 40° C. for 12 to 24 h.

The compounds represented by Formula I provided by the present invention can be used for preventing and/or treating CypA-mediated diseases, such as CypA-mediated inflammation, autoimmune diseases and/or tumors;
preferably, the inflammation comprises pneumonia;
the autoimmune diseases comprise rheumatoid arthritis, systemic lupus erythematosus and psoriasis;
the tumors comprise lung tumors.

Also within the scope of the present invention is the use of the compound represented by Formula I for any of the following functions or for preparing a product having any of the following functions:
1) degrading the CypA protein;
2) alleviating influenza virus-induced pneumonia;
3) treating rheumatoid arthritis;
4) inhibiting the migration and infiltration of cancer cells.

The present invention further provides a method for treating CypA-mediated diseases, such as CypA-mediated inflammation, autoimmune diseases and/or tumors, in a subject, the method comprising administering to the subject a therapeutically effective amount of the compound represented by Formula I;

the method comprises administering to the subject a therapeutically effective amount of the compound represented by Formula I.

The present invention further provides a pharmaceutical composition comprising the compound represented by Formula I as an active ingredient and at least one pharmaceutically acceptable carrier, excipient and/or diluent.

### BRIEF DESCRIPTION OF THE DRAWINGS

FIG. 1 shows a synthetic scheme for the compound represented by Formula I of the present invention.

FIG. 2 shows the effect of the compound represented by Formula I on cell activity.

FIGS. 3A-3B show the results of the degradation activity of the compound represented by Formula I on CypA, where FIG. 3A shows the results of the degradation of CypA by the compound represented by Formula I at different concentrations, and FIG. 3B shows the grayscale analysis of the relative expression of CypA in FIG. 3A.

FIGS. 4A-4H show the results of the alleviation of influenza B virus-induced pneumonia by the compound represented by Formula I, where FIG. 4A shows the detection of the expression of CypA in mice of each group, FIG. 4B shows the lung index of mice in each group, FIG. 4C shows the pathology of mice in each group, FIG. 4D shows the pathological scores of mice in each group, FIG. 4E shows the relative expression of Il1b mRNA in mice of each group, FIG. 4F shows the relative expression of Tnfa mRNA in mice of each group, FIG. 4G shows the relative expression of Il6 mRNA in mice of each group, and FIG. 4H shows the relative expression of Ifng mRNA in mice of each group.

FIGS. 5A-5D show the results of the inhibition of rheumatoid arthritis by the compound represented by Formula I, where FIG. 5A shows the HE images of the ankles of rats in each group, FIG. 5B shows the joint swelling rate of rats in each group, FIG. 5C shows the joint index of rats in each group, and FIG. 5D shows the ankle pathological scores of rats in each group.

FIGS. 6A-6B show the results of the inhibition of the migration and infiltration of lung cancer cells by the compound represented by Formula I, where FIG. 6A shows the results of the observation of the migration and infiltration of cells in each group by the Traswell method, and FIG. 6B shows the cell counts in FIG. 6A.

### DETAILED DESCRIPTION OF THE INVENTION

The experimental methods used in the following examples are conventional, unless otherwise specified.

The materials, reagents, etc. used in the following examples are all commercially available, unless otherwise specified.

### Example 1. Preparation of a Compound Represented by Formula I for Targeting and Degrading CypA

The synthetic scheme is shown in FIG. 1.

### 1. Synthesis of Compound 2

Compound 1 (1.0 g, 3.2 mmol), KOH (0.64 g, 11.4 mmol) and H₂O (0.4 mL, 22 mmol) were added to BnOH (10 mL, 22 mmol), and the reaction solution was stirred with a microwave tube at 130° C. overnight. H₂O (12 mL) was added to the reaction solution, then filtered and dried to obtain compound 2 (0.24 g, 22%) as a white solid.

### 2. Synthesis of Compound 4

Compound 2 (1.53 g, 4.6 mmol) and compound 3 (0.6 mL, 5.0 mmol) were added to toluene (20 mL), and the reaction solution was stirred at 110° C. overnight. The reaction solution was extracted with ethyl acetate (3×100 mL). The organic phase was washed with saturated sodium chloride, dried over anhydrous sodium sulfate and concentrated. The crude product was separated by column chromatography (PE:EA=3:10) to obtain compound 4 (0.9 g, 42%) as a yellow oil.

### 3. Synthesis of Compound 5

Compound 4 (0.8 g, 1.74 mmol) and Pd/C (10 wt %, 1.6 g) were added to methanol (10 mL) and stirred under a hydrogen atmosphere at 25° C. overnight. The reaction solution was filtered through diatomaceous earth and concentrated. The crude product was purified by column chromatography (PE:EA=3:10) to obtain compound 5 (0.3 g, 61%) as a white solid.

### 4. Synthesis of Compound 8

Compound 6 (0.63 g, 2.89 mmol) was added to DCM (10 mL), followed by compound 7 (0.82 g, 4.33 mmol), Et₃N (0.8 mL, 5.78 mmol) and DMAP (35 mg, 0.29 mmol) in sequence at 0° C. The reaction solution was stirred at 25° C. overnight. After completion of the reaction, the reaction solution was extracted with ethyl acetate (2×100 mL). The organic phase was washed with saturated sodium chloride, dried over anhydrous sodium sulfate and concentrated. The crude product was separated by column chromatography (PE:EA=1:4) to obtain compound 8 (1.03 g, 96%) as a colorless oil.

### 5. Synthesis of Compound 9

Compound 5 (0.1 g, 0.36 mmol), compound 8 (0.12 g, 0.32 mmol) and KOH (0.15 g, 1.08 mmol) were added to DMF (5 mL). The reaction solution was stirred at 25° C. overnight. After completion of the reaction, the reaction solution was extracted with ethyl acetate (3× 20 mL). The organic phase was washed with saturated sodium chloride, dried over anhydrous sodium sulfate and concentrated. The crude product was separated by column chromatography (PE:EA=1:4) to obtain compound 9 (30 mg, 17%) as a colorless oil.

### 6. Synthesis of Compound 10

Compound 9 (30 mg, 0.06 mmol) was added to DCM (2 mL), followed by TFA (0.4 mL). The reaction solution was stirred at room temperature for 0.5 h. After completion of the reaction, the reaction solution was directly concentrated to obtain compound 10 (25 mg, 96%) as a colorless oil.

### 7. Synthesis of the Compound Represented by Formula I

Compound 10 (0.13 g, 0.3 mmol) was dissolved in DMF (5 mL), and HOBT (48 mg, 0.36 mmol), EDCI (68 mg, 0.36 mmol) and DIEA (0.15 mL, 0.9 mmol) were added in sequence. The reaction solution was stirred at 25° C. for 0.5 h, and compound 11 (0.17 g, 0.36 mmol) was added at 0° C. The reaction solution was then stirred at 25° C. overnight. After completion of the reaction, the reaction solution was extracted with ethyl acetate (3× 50 mL). The organic phase was washed with saturated sodium chloride, dried over anhydrous sodium sulfate and concentrated. The crude product was purified by Prep-HPLC to obtain compound I (0.1 g, 38%) as a white solid. The structural characterization data of compound I were as follows:

¹HNMR (400 MHZ, CD₃OD) δ 9.03 (s, 1H), 7.47-7.35 (m, 5H), 6.60 (dd, J=13.1, 8.4 Hz, 2H), 4.98 (dd, J=14.0, 7.0 Hz, 2H), 4.68 (m, J=4.7 Hz, 1H), 4.61-4.53 (m, 1H), 4.43 (br, 1H), 4.23 (t, J=6.2 Hz, 2H), 4.06-3.90 (m, 2H), 3.84 (d, J=11.2 Hz, 1H), 3.74 (dd, J=11.3, 3.8 Hz, 2H), 3.61 (t, J=6.1 Hz, 2H), 2.49 (s, 3H), 2.19 (m, 1H), 2.05-1.90 (m, 6H), 1.85-1.59 (m, 9H), 1.49 (d, J=7.0 Hz, 3H), 1.47-1.24 (m, 6H), 1.01 (s, J=6.7 Hz, 9H).

### Example 2. Assay for the Effect of the Compound Represented by Formula I on A549 Cell Activity by CCK-8 Method

I. Treatment of A549 Cells with the Compound Represented by Formula I

(1) A549 cells in the logarithmic phase were collected, the concentration of the cell suspension was adjusted, and 100 μL was added per well. The cells to be tested were plated to a density of 10³ to 10⁴ cells/well and incubated at 37° C. under 5% CO₂ until the bottom of the wells was covered by a single layer of cells.

(2) Different concentrations of the compound represented by Formula I were added: 0, 10, 10², 10³, 10⁴ and 10⁵ nM/well, with 3 replicate wells set, and incubated at 37° C. under 5% CO₂ for 12 hours.

### II. Detection of A549 Cell Activity by CCK-8 Method

(1) 10 μL CCK-8 reagent was added to the above cells in each well and reacted for 2 h.

(2) The incubation was terminated, and the culture medium in the wells was carefully aspirated.

(3) The absorbance of each well was measured using a microplate reader with a wavelength of 450 nm.

(4) The absorbance ratio between the experimental group and the control group was calculated as follows: Cell viability=(Absorbance of the experimental group−Blank control)+ (Absorbance of the control group−Blank control)×100%.

The effect of the compound in the example of the present invention on A549 cell activity was as follows:

The compound represented by Formula I was less toxic to A549 cells at concentrations ranging from 0 to 10⁵ nM, and the cell viability remained above 80%, as shown in FIG. 1.

### Example 3. Biological Activity Assay of the Compound Represented by Formula I at the Level of Western Blot Experiment

I. Treatment of A549 Cells with the Compound Represented by Formula I

(1) A549 cells in the logarithmic phase were collected, the concentration of the cell suspension was adjusted, and 100 μL was added per well. The cells to be tested were plated to a density of 10³ to 10⁴ cells/well and incubated at 37° C. under 5% CO₂ until the bottom of the wells was covered by a single layer of cells.

(2) Different concentrations of the compound represented by Formula I were added: 0, 10, 10², 10³, 10⁴ and 10⁵ nM/well, with 3 replicate wells set, and incubated at 37° C. under 5% CO₂ for 12 hours.

### II. Protein Samples Collection

(1) The treated cells were scraped off from the culture medium, suspended thoroughly, and then collected by centrifugation at 300 g for 5 minutes. After washing with PBS once, the PBS was discarded.

(2) 100 μL of 2× Loading Buffer was added to each sample, shaken thoroughly to mix well, denatured at 100° C. for 5 minutes, and stored at −20° C. after mixing well or directly used for Western Blot detection. The Formula of 5× Loading Buffer was as follows: 250 mM Tris-HCl (pH 6.8), 10% (W/V) SDS, 0.5% (W/V) bromophenol blue, 50% (V/V) glycerol, 5% (W/V) β-mercaptoethanol (2-ME). 2× Loading Buffer was prepared by adding 1.5 times the volume of double distilled water to 5× Loading Buffer.

(3) The specific steps for Western Blot (WB) detection were as follows:

1) An appropriate concentration of SDS-PAGE gel was prepared: the concentration of a separating gel of 10% and the concentration of a stacking gel of 5%.

2) Sample preparation: protein samples were prepared according to experimental requirements, centrifuged, mixed well, and loaded into the loading wells of SDS-PAGE gel. The loading volume was adjusted appropriately according to the protein quantification results.

3) Electrophoresis: the power was turned on, and the protein samples were run at 80 volts in the stacking gel. When the protein samples entered the separating gel, the voltage was adjusted to 120 volts to continue the electrophoresis. The electrophoresis was terminated when bromophenol blue almost completely ran out of the PAGE gel.

4) Membrane transfer: after the electrophoresis was completed, the gel was removed, and a membrane transfer device was installed in the following order: (negative electrode), filter paper, gel, activated PVDF membrane, filter paper, (positive electrode). Then, the transfer device was clamped and placed in a membrane transfer buffer, and finally placed in an ice box and placed in a 4° C. refrigerator at a constant voltage of 100V for 40 minutes.

5) Blocking: after the membrane transfer was completed, the PVDF membrane was removed, immersed in TBST buffer containing 5% skimmed milk powder, and shaken on a shaker at room temperature for 1 hour.

6) Primary antibody incubation: after the blocking was completed, the membrane was washed 3 times with TBST buffer, and then an appropriately diluted primary antibody was added and incubated at 4° C. overnight. The PVDF membrane was shaken and washed 3 times with TBST buffer for 5 minutes each time.

7) Secondary antibody incubation: the TBST buffer was discarded, and the diluted secondary antibody was added and shaken on a shaker at room temperature for 1 hour. The secondary antibody was discarded, and the PVDF membrane was shaken and washed 3 times with TBST buffer for 5 minutes each time.

8) Exposure: an ECL chromogenic substrate was evenly spread over the PVDF membrane to expose for imaging.

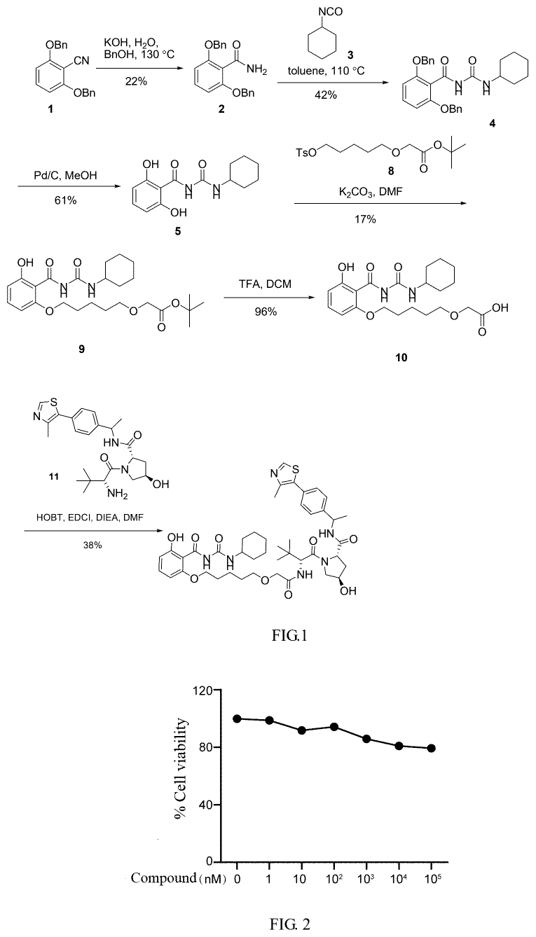

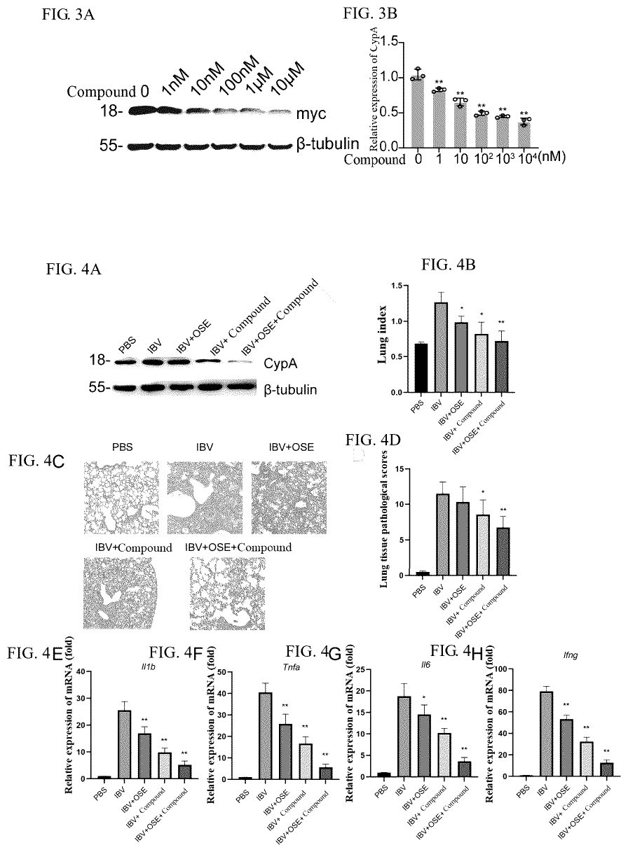

The degradation activity of the compound represented by Formula I of the present invention on CypA was as follows: in the A549 cell line, the degradation effect of the compound represented by Formula I on the CypA protein could be clearly observed in the WB results, as shown in FIG. 2, and the half-degradation concentration of the compound represented by Formula I on CypA was below 100 nM.

### Example 4. Protection Effect of the Compound Represented by Formula I in IBV-Induced Pneumonia

### I. Grouping of Mice and Construction of Mouse Pneumonia Model

Female C57BL/6 mice aged 6 to 8 weeks were randomly divided into 5 groups: blank group (PBS), untreated group (IBV), treatment group 1 (IBV+compound), treatment group 2 (IBV+OSE (oseltamivir, an anti-influenza virus drug)), treatment group 3 (IBV+compound+OSE), with 5 mice in each group and a body weight of 19±3 g. C57 BL/6 mice were anesthetized by intraperitoneal injection of Avertin at 200 μL/10 g of body weight. After the mice were anesthetized, 50 μL of PBS was dripped into the nasal cavity of mice in the blank group, and 50 μL of IBV-/Guangxi-Jiangzhou/1352/2018 (8000 PFU) resuspended in PBS was dripped into the nasal cavity of mice in the untreated group, treatment group 1, treatment group 2 and treatment group 3. The mice in treatment group 1 were injected with the compound (10 mg/kg of body weight/day, dissolved in 100 μL of PBS) through the tail vein 24 h after IBV infection. The mice in treatment group 2 were intraperitoneally injected with 0.2 mg of OSE (dissolved in 200 μL of normal saline) at a dose of 10 mg/kg 24 h after IBV infection, and 0.2 mg of OSE was injected once every 24 h. The mice in treatment group 3 were intraperitoneally injected with the compound (10 mg/kg of body weight/day, dissolved in 100 μL of PBS) and 0.2 mg of OSE (dissolved in 200 μL of normal saline) at a dose of 10 mg/kg 24 h after IBV infection, and 0.2 mg of OSE was injected once every 24 h. The blank and untreated groups were given the same dose of normal saline according to the injection time of treatment group 2. The mice in each group were sacrificed 7 days after IBV infection, and subsequent detection experiments were performed.

### II. Detection of CypA Expression in Lung Tissue

(1) Approximately 100 mg of lung tissue from mice in each group was taken, placed in a lysis buffer, and lysed in a tissue crusher at a rotational speed of 4000 rpm for 40 s, with an interval of 10 s, and the process was run twice.

(2) After the run was completed, the product was centrifuged at 12000 rpm at 4° C. for 15 min. The supernatant was taken, added with 5× loading buffer, and incubated in a metal bath for 10 min for detecting CypA expression by WB experiment.

(3) The WB experiment method was the same as in Example 3.

### III. Lung Index Detection

The mice were anesthetized with ether and weighed. The blood of the mice was collected completely, and the mice were fixed in a supine position. The chest cavity was opened, the esophagus and heart were removed, and the lung tissue was isolated and weighed. The lung index was calculated by weighing the lung of mice: Lung index=Mouse lung weight-Mouse body weight. The lung index is a very important indicator in determining the severity of pulmonary inflammation, and inflammation will lead to an increase in the lung index.

### IV. Pathological Observation of Mouse Lung Tissue

(1) The lung tissue of mice was fixed with 4% paraformaldehyde for more than 16 hours, followed by conventional dehydration, wax immersion, embedding, sectioning and conventional HE staining.

(2) The sample was scored separately according to four indicators: pulmonary interstitial edema, alveolar edema, inflammatory cell infiltration, alveolar hemorrhage and hyaline membrane formation, as follows: 0 for none; 1 for mild; 2 for moderate; 3 for extensive, and the cumulative sum of the scores was the tissue score.

### V. Detection of Cytokine Expression in Lung Tissue

(1) Approximately 100 mg of lung tissue from mice in each group was taken, placed in 1 mL of Trizol, and lysed in a tissue crusher at a rotational speed of 4000 rpm for 40 s, with an interval of 10 s, and the process was run twice.

(2) After the run was completed, the total mRNA was extracted by the TRIZOL method and reverse transcribed to obtain cDNA for qPCR detection.

(3) qPCR Reaction Procedure:

\textcircled{1} 95° C. for 30 s; \textcircled{2} 95° C. for 30 s; \textcircled{3} 60° C. for 60 s; 2 and 3 were repeated for 40 cycles. The fold change was calculated using the 2^{-ΔΔCT} method.

### VI. Experimental Results

The experimental results are shown in FIG. 4, where panel A shows the detection of the expression of CypA in mice of each group, panel B shows the lung index of mice in each group, panel C shows the pathology of mice in each group, panel D shows the pathological scores of mice in each group, panel E shows the relative expression of Il1b mRNA in mice of each group, panel F shows the relative expression of Tnfa mRNA in mice of each group, panel G shows the relative expression of Il6 mRNA in mice of each group, and panel H shows the relative expression of Ifng mRNA in mice of each group.

(1) As can be seen from panel A in FIG. 4, the compound represented by Formula I could significantly reduce the expression of CypA in mouse lung tissue.

(2) As can be seen from panel B in FIG. 4, panel C in FIG. 4 and panel D in FIG. 4, the IBV+compound group and the IBV+OSE+compound group could significantly alleviate lung injury in mice. The IBV+OSE+compound group had a better effect.

(3) As can be seen from panel E in FIG. 4, panel F in FIG. 4, panel G in FIG. 4 and panel H in FIG. 4, the IBV+compound group and the IBV+OSE+compound group could significantly reduce the expression of IL-1B, TNF-α, IL-6 and IFN-γ in the lung tissue of mice, thereby alleviating lung inflammation in mice. The IBV+OSE+compound group had a better effect.

### Example 5. Protection Effect of the Compound Represented by Formula I in Rheumatoid Arthritis

### I. Grouping of Rats and Construction of Rat Arthritis Model

The BCG vaccine was inactivated in a water bath at 80° C. for 1 h, ground thoroughly with incomplete Freund adjuvant, and mixed well to prepare 15 mg/mL Freund complete adjuvant (FCA). Lewis rats were divided into 4 groups: blank group (PBS), untreated group (UT), treatment group 1 (compound) and treatment group 2 (MTX (methotrexate)), with 5 rats in each group and a body weight of 19±3 g. Except for the PBS group, rats were intradermally injected with 0.1 mL of FCA in the right hind paw. On day 14 after injection, rats in treatment group 1 (compound) were injected with the compound (10 mg/kg of body weight, dissolved in 100 μL of PBS) through the tail vein three times/week; rats in treatment group 2 (MTX (methotrexate)) were intraperitoneally injected with methotrexate (1.2 mg/kg of body weight) three times/week.

### II. Joint Swelling Rate

The circumference of the metatarsophalangeal joint was measured. The circumference of the ankle joint of the left hind paw (on the secondary lesion side) of all rats was measured once before modeling, before administration and 3 weeks after administration, respectively, and the percentage of joint swelling was calculated.

### III. Joint Index

The joint index was determined by joint swelling score before administration (Day 0) and 3 weeks after administration. Scoring was carried out based on the redness and swelling degree of the ankle and toe joints of the front and rear limbs of rats and the affected joint index, as follows: 0 for normal; 1 for the redness and swelling of one joint; 2 for the redness and swelling of two or more joints; 3 for severe redness and swelling of the paw below the ankle joint; 4 for the redness and swelling of the entire paw including the ankle joint and inability to bear weight. The limbs were scored separately, and the cumulative sum of the scores was the joint index.

### IV. Pathological Examination of Joint Synovium

(1) On the day 21 of administration, rats were euthanized after blood collection. The left metatarsophalangeal joint was fixed with 4% paraformaldehyde for more than 16 hours, followed by decalcification with 10% EDTA, conventional dehydration, wax immersion, embedding, sectioning and conventional HE staining.

(2) Joint synovial proliferation and the degree of sub-synovial inflammation were observed under a light microscope, and synovial cell proliferation was scored. The synovial cells of normal synovial tissue were monolayer, arranged well, and had a flat shape, with a small amount of inflammatory cell infiltration, without vascular proliferation, fibrosis or papillary proliferation (scored as 0). In cases of synovial cell proliferation, the number of cell layers slightly increased, fibrous tissue proliferation occurred in the synovial tissue, the capillaries increased, dilated and congested, and inflammatory cell and fibroblast infiltration could be seen (graded and scored from 1-3 depending on the severity).

### V. Experimental Results

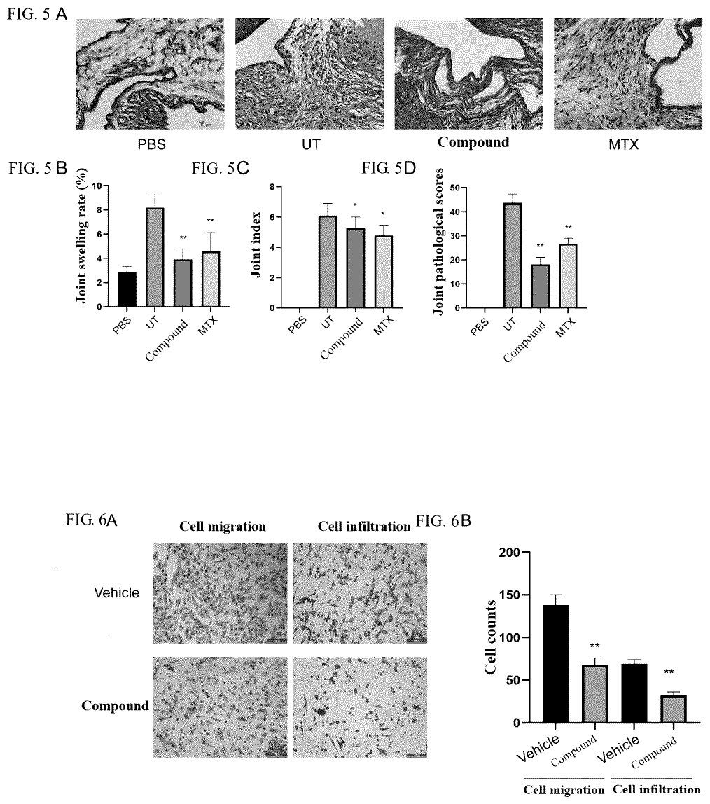

As shown in FIG. 5, where panel A shows the HE images of the ankles of rats in each group, panel B shows the joint swelling rate of rats in each group, panel C shows the joint index of rats in each group, and panel D shows the ankle pathological scores of rats in each group.

As can be seen from the panels in FIG. 5, the compound represented by Formula I of the present invention could significantly relieve joint swelling in rats, reduce the joint index of the ankle joint, and alleviate the inflammation of the ankle joint.

### Example 6. Inhibition of the Compound Represented by Formula I on the Migration and Infiltration of Lung Cancer Cells

### I. Cell Treatment

(1) A549 cells in the logarithmic phase were collected, the concentration of the cell suspension was adjusted, and 100 μL was added per well. The cells to be tested were plated to a density of 10³-10⁴ cells/well and incubated at 37° C. under 5% CO₂ until the bottom of the wells was covered by a single layer of cells.

(2) Different concentrations of the compound represented by Formula I were added: 0, 10, 10², 10³, 10⁴ and 10⁵ nM/well, with 3 replicate wells set, and incubated at 37° C. under 5% CO₂ for 12 hours.

The upper chamber of a 24-well cell culture insert (8 μmol/L pore size) was coated (no migration) or (invasive) with a matrix (1 mg/mL) and dried at 37° C. for 30 min.

(3) A549/WT and A549/CypA-cells were trypsinized, washed and suspended in serum-free DMEM after treatment with 20 ng/ml TGF-β for 48 h.

### II. Determination by Transwell Chambers (Corning Inc.)

(1) DMEM containing 10% fetal bovine serum was added to the lower chamber per approximately 5×10⁴ cells inserted in the upper chamber.

(2) After incubation for 6 h (migration) or 24 h (invasion), non-migrating or invasive cells residing on the upper part of the insert membrane were removed with a cotton swab.

(3) The cells that migrated to the underside of the membrane were fixed and stained with 2% crystal violet in ethanol.

(4) The stained cells were observed and counted under a Japan Olympus CKX41 microscope.

### III. Experimental Results

As shown in FIG. 6, where panel A shows the migration and infiltration of cells in each group by the Traswell method, and panel B shows the cell counts of panel A in FIG. 6.

As can be seen from FIG. 5, the compound represented by Formula I of the present invention could significantly reduce the migration and infiltration of lung cancer cells. The present invention further comprises the following preferred embodiments:

1. Use of the compound represented by Formula I as described herein for preventing and/or treating CypA-mediated diseases.

2. Use of the compound represented by Formula I as described herein for preventing and/or treating CypA-mediated inflammation, autoimmune diseases and/or tumors.

3. Use of the compound represented by Formula I as described herein for preventing and/or treating CypA-mediated pneumonia.

4. Use of the compound represented by Formula I as described herein for preventing and/or treating CypA-mediated rheumatoid arthritis, systemic lupus erythematosus and psoriasis.

5. Use of the compound represented by Formula I as described herein for preventing and/or treating CypA-mediated lung tumors.

6. Use of the compound represented by Formula I as described herein for degrading the CypA protein, alleviating influenza virus-induced pneumonia, treating rheumatoid arthritis, and inhibiting the migration and infiltration of cancer cells.

7. The compound of Formula I as described herein for treating CypA-mediated diseases.

8. The compound of Formula I according to embodiment 7, wherein the CypA-mediated diseases are CypA-mediated inflammation, preferably CypA-mediated pneumonia and CypA-mediated rheumatoid arthritis.

9. The compound of Formula I as described herein for treating tumors.

10. The compound of Formula I according to embodiment 9, wherein the tumors are lung tumors.

11. The compound of Formula I as described herein for treating autoimmune diseases.

12. The compound of Formula I according to embodiment 11, wherein the autoimmune diseases comprise systemic lupus erythematosus and psoriasis.

13. A method for treating CypA-mediated diseases in a subject, the method comprising administering to the subject a therapeutically effective amount of the compound of Formula I as described herein.

14. A method for treating tumors in a subject, the method comprising administering to the subject a therapeutically effective amount of the compound of Formula I as described herein.

15. A method for treating autoimmune diseases in a subject, the method comprising administering to the subject a therapeutically effective amount of the compound of Formula I as described herein.

### INDUSTRIAL APPLICATIONS

The compound represented by Formula I provided by the present invention can target and degrade the CypA protein, and thus can be used for preparing a drug for treating inflammation, autoimmune diseases, tumors and other related diseases. The compound represented by Formula I of the present invention has the function of significantly inhibiting virus-induced pneumonia, rheumatoid arthritis and lung cancer cell migration and infiltration.

1. A compound represented by Formula I:

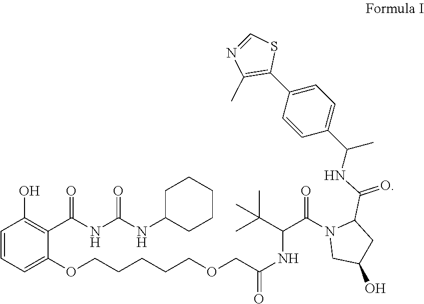 [SMILES: Cc1ncsc1-c1ccc(C(C)NC(=O)C2C[C@@H](O)CN2C(=O)C(NC(=O)COCCCCCOc2cccc(O)c2C(=O)NC(=O)NC2CCCCC2)C(C)(C)C)cc1]

2. A preparation method of the compound represented by Formula I according to claim 1, comprising the steps of:
S1, subjecting a compound represented by Formula 1 to a hydrolysis reaction in the presence of a basic compound A to obtain a compound represented by Formula 2; 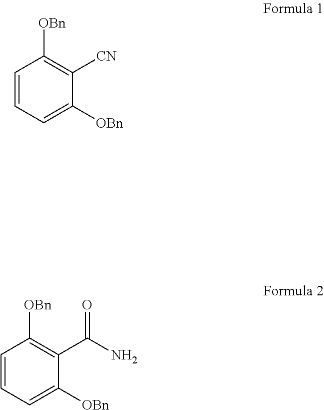
wherein Bn represents benzyl;
S2, subjecting the compound represented by Formula 2 to a nucleophilic addition reaction with a compound represented by Formula 3 to obtain a compound represented by Formula 4; 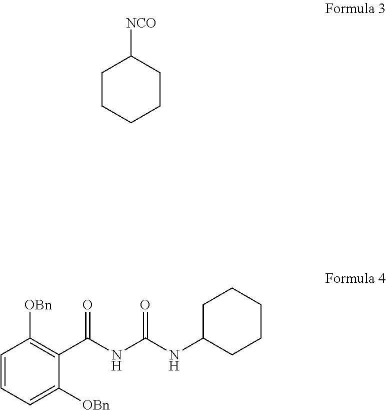
S3, subjecting the compound represented by Formula 4 to a reduction reaction by palladium on carbon to obtain a compound represented by Formula 5; 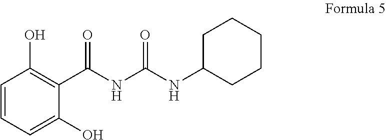 [SMILES: O=C(NC(=O)c1c(O)cccc1O)NC1CCCCC1]
S4, subjecting a compound represented by Formula 6 to a nucleophilic substitution reaction with a compound represented by Formula 7 in the presence of triethylamine and DMAP to obtain a compound represented by Formula 8; 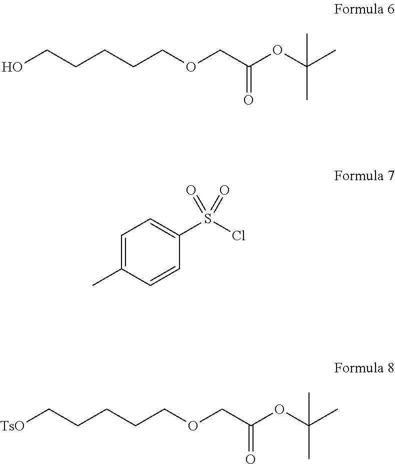
S5, subjecting the compound represented by Formula 5 to a nucleophilic substitution reaction with the compound represented by Formula 8 in the presence of K₂CO₃ to obtain a compound represented by Formula 9; 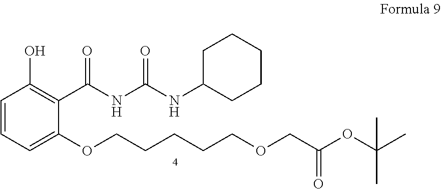 [SMILES: CC(C)(C)OC(=O)COCCCCCOc1cccc(O)c1C(=O)NC(=O)NC1CCCCC1]
S6, subjecting the compound represented by Formula 9 to a deprotection reaction in the presence of trifluoroacetic acid to obtain a compound represented by Formula 10; and 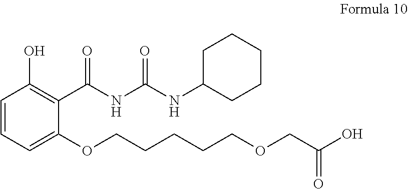 [SMILES: O=C(O)COCCCCCOc1cccc(O)c1C(=O)NC(=O)NC1CCCCC1]
S7, subjecting the compound represented by Formula 10 to an amidation reaction with a compound represented by Formula 11 in the presence of HOBT, EDCI and DIEA to obtain the compound represented by Formula I according to claim 1;

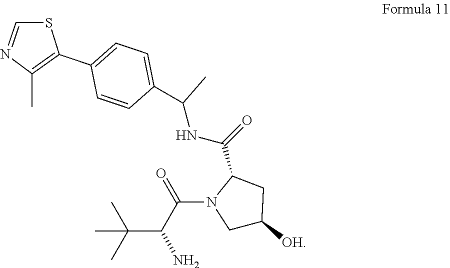 [SMILES: Cc1ncsc1-c1ccc(C(C)NC(=O)[C@@H]2C[C@@H](O)CN2C(=O)[C@H](N)C(C)(C)C)cc1]

3. The preparation method according to claim 2, wherein in step S1, a solvent used in the hydrolysis reaction is benzyl alcohol;
the molar ratio of the compound represented by Formula 1 to the benzyl alcohol is 1:5 to 10;
the temperature of the hydrolysis reaction is 100 to 150° C., and the time of the hydrolysis reaction is 12 to 24 h;
in step S2, the temperature of the nucleophilic addition reaction is 100 to 150° C., and the time of the nucleophilic addition reaction is 12 to 24 h;
in step S3, the mass percentage of palladium in the palladium on carbon is 10%;
the reduction reaction is carried out in methanol.

4. The preparation method according to claim 2, wherein in step S4, the molar ratio of the compound represented by Formula 6 to the compound represented by Formula 7 is 1:1 to 1.5;
the molar ratio of the triethylamine to the compound represented by Formula 6 is 1:1 to 1.5;
the molar ratio of the DMAP to the compound represented by Formula 6 is 1:10 to 15;
the temperature of the nucleophilic substitution reaction is 10 to 40° C., and the time of the nucleophilic substitution reaction is 12 to 24 h;
in step 5, the temperature of the nucleophilic substitution reaction is 10 to 40° C., and the time of the nucleophilic substitution reaction is 12 to 24 h.

5. The preparation method according to claim 4, wherein in step S6, the temperature of the deprotection reaction is 10 to 40° C., and the time of the deprotection reaction is 1 to 2 h; in step S7, after adding the HOBT, the EDCI and the DIEA to the compound represented by Formula 10, stirring at 10 to 40° C. for 1 to 2 h, adding the compound represented by Formula 11 at 0° C. and then reacting at 10 to 40° C. for 12 to 24 h.

6. Use of the compound represented by Formula I according to claim 1 in the preparation of a drug for preventing and/or treating CypA-mediated diseases.

7. The use according to claim 6, wherein the CypA-mediated diseases comprise inflammation, autoimmune diseases and/or tumors.

8. The use according to claim 7, wherein the inflammation comprises pneumonia;
the autoimmune diseases comprise rheumatoid arthritis, systemic lupus erythematosus and psoriasis;
the tumors comprise lung tumors.

9. Use of the compound represented by Formula I according to claim 1 for preparing a product having any of the following functions:
1) degrading CypA protein;
2) alleviating influenza virus-induced pneumonia;
3) treating rheumatoid arthritis;
4) inhibiting the migration and infiltration of cancer cells.

10. A pharmaceutical composition comprising the compound represented by Formula I according to claim 1 as an active ingredient and at least one pharmaceutically acceptable carrier, excipient and/or diluent.

11. A method for treating CypA-mediated diseases in a subject, the method comprising administering to the subject a therapeutically effective amount of the compound represented by Formula I according to claim 1.

12. The method according to claim 11, wherein the CypA-mediated diseases comprise inflammation, autoimmune diseases and/or tumors.

13. The method according to claim 12, wherein the inflammation comprises pneumonia;
the autoimmune diseases comprise rheumatoid arthritis, systemic lupus erythematosus and psoriasis;
the tumors comprise lung tumors.

14. The compound represented by Formula I according to claim 1 for treating CypA-mediated diseases.

15. The compound represented by Formula I according to claim 14, wherein the CypA-mediated diseases comprise inflammation, autoimmune diseases and/or tumors.

16. The compound represented by Formula I according to claim 15, wherein the inflammation comprises pneumonia;
the autoimmune diseases comprise rheumatoid arthritis, systemic lupus erythematosus and psoriasis;
the tumors comprise lung tumors.
